(19) United States
(12) Patent Application Publication
(10) Pub. No.: US 2025/0174327 A1
(43) Pub. Date: May 29, 2025

(54) SYNTHETIC CONTROLS FOR SURVIVAL DATA

(71) Applicant: Microsoft Technology Licensing, LLC, Redmond, WA (US)

(72) Inventors: Javier GONZÁLEZ HERNÁNDEZ, Cambridge (GB); Alicia Marie CURTH, Cambridge (GB); Hoifung POON, Bellevue, WA (US); Aditya Vithal NORI, Cambridge (GB)

(21) Appl. No.: 18/592,455

(22) Filed: Feb. 29, 2024

Related U.S. Application Data
(60) Provisional application No. 63/604,175, filed on Nov. 29, 2023.

(51) Int. Cl. G16H 20/10; A61B 5/00

(52) U.S. Cl. CPC G16H 20/10; A61B 5/4848

(57) ABSTRACT

A computerized system and method for creating synthetic controls in survival analysis is provided. A target group of patients who are administered a drug and a control group of patients who are not administered the drug are created from real data of patients. A weight is applied to a common feature of each patient in the control group of patients so that a linear combination of the common feature of the patients in the control group of patients becomes similar to a particular patient in the target group of patients. A synthetic patient is created for each patient in the control group of patients. Because the common feature of the synthetic patient is similar to the particular patient in the target group, an efficacy of the drug may be determined by comparing the target group of patients with the synthetic patient.

### CROSS-REFERENCE TO RELATED APPLICATION

This application claims priority to U.S. Provisional Patent Application No. 63/604,175, entitled “SYNTHETIC CONTROLS FOR SURVIVAL DATA”, filed on Nov. 29, 2023, the disclosure of which is incorporated herein by reference in its entirety.

### BACKGROUND

Synthetic control (SC) methods have gained popularity in economics recently, where they have been applied in the context of inferring the effects of actions on standard continuous outcomes. In medical trial applications, survival outcomes are of primary interest, and to obtain data on the effects of drugs and/or other treatments, control groups are used. This means that a subset of the eligible patients is not given the treatment in order to form a control group. However, in some cases, all eligible patients would prefer to be given the treatment, and potential benefits to those patients must be weighed against the value of collecting data associated with a control group of patients who have been given placebo or otherwise not given the treatment being examined.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

A computerized method for creating synthetic controls in survival analysis is described. A target group of patients and a control group of patients are created from data associated with a plurality of patients, wherein the target group of patients comprises patients to whom a drug is to be administered, the control group of patients comprises patients to whom the drug is not administered, and wherein each patient in the target group of patients and the control group of patients have a common feature. A weight is applied to the common feature of each patient in the control group of patients so that a linear combination of the common feature of the patients in the control group of patients becomes similar to a particular patient in the target group of patients. Applying the weight comprises minimizing a distanced between the common feature of the particular patient in the target group of patients and the linear combination of the common feature of the patients in the control group of patients, wherein the distance is minimized at least in part by penalizing the distance using a variance penalty. A synthetic patient is created for each patient in the control group of patients, wherein the synthetic patients have common features similar to the particular patient in the target group of patients.

### BRIEF DESCRIPTION OF THE DRAWINGS

The present description will be better understood from the following detailed description read considering the accompanying drawings, wherein:

FIG. 1 is a block diagram illustrating synthetic control arms as a solution for creating synthetic controls in survival analysis;

FIG. 2A is a block diagram illustrating an example implementation generating synthetic control arms from aggregate inputs;

FIG. 2B is a block diagram illustrating an example implementation generating synthetic control arms from patient-level inputs;

FIG. 3A illustrates an example graph showing that interpolation bias is high on non-linear part of the exponential;

FIG. 3B illustrates an example graph showing censoring bias;

FIG. 3C illustrates an example graph showing initialization effects of bias;

FIG. 3D illustrates performance of mean absolute error (MAE) of predicting the Restricted Mean Survival Time (RMST).

FIG. 4 illustrates an example flow chart implementing aspects of the disclosure for creating synthetic controls in survival analysis; and

FIG. 5 illustrates an example computing apparatus as a functional block diagram for executing the instructions illustrated in FIG. 4.

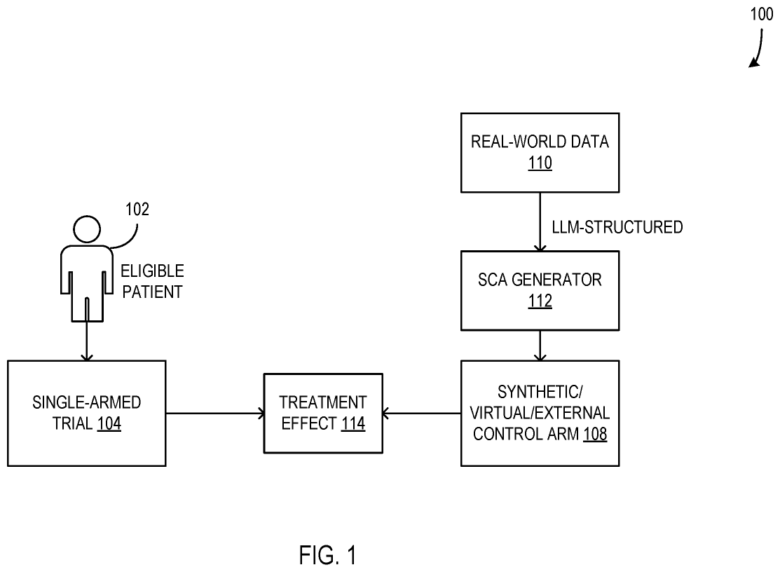

Corresponding reference characters indicate corresponding parts throughout the drawings. In FIGS. 1 to 5, the systems are illustrated as schematic drawings. The drawings may not be to scale. Any of the figures may be combined into a single example or embodiment.

### DETAILED DESCRIPTION

Usually, for clinical trials, each eligible patient is randomly selected into a target group in which the patient will receive the new treatment, or in a control group in which the patient will receive the standard of care treatment (e.g., a placebo). However, terminally ill patients may agree to receive the new treatment (e.g., with a new drug) and they may refuse to be part of the control group.

Examples of the disclosure create synthetic controls, including creating synthetic patients from the patients in the target group for clinical trials. For example, all the patients may be administered the drug (e.g., all patients are selected into the target group), and efficacy of the drug may be determined by comparing the target group of patients who are administered the drug with the synthetic patients who are not administered the drug. Thus, examples of the disclosure advantageously reduce the number of patients required for clinical trials of a new drug. Further, all the terminally ill patients may be administered the drug, and the efficacy of the drug may still be determined by comparison with the synthetic patients. Examples of the disclosure reduce the computational processing time for determining efficacy of the drug as the number of patients is reduced.

In some examples, aspects of the disclosure provide a computerized method for creating synthetic controls in survival analysis by: creating a target group of patients and a control group of patients from data (e.g., real data obtained from a hospital) associated with a plurality of patients, the target group of patients comprising patients to whom a drug is to be administered, the control group of patients comprising patients to whom the drug is not administered, each patient in the target group of patients and the control group of patients having a common feature; applying a weight to the common feature of each patient in the control group of patients so that a linear combination of the common feature of the patients in the control group of patients becomes similar to a particular patient in the target group of patients, applying the weight comprising: minimizing a distance between the common feature of the particular patient in the target group of patients and the linear combination of the common feature of the patients in the control group of patients, the distance being minimized by penalizing the distance using a variance penalty; and creating a synthetic patient for each patient in the control group of patients, the synthetic patient having the common feature similar to the particular patient in the target group of patients.

The common feature represents characteristics of the patient, and the weight is applied to the common feature such that the linear combination of the common feature of the patients in the control group of patients becomes similar to the particular patient in the target group of patients. The characteristics of the patient may include, but are not limited to, age, menopausal status, tumor size, tumor differentiation grades, number of positive lymph nodes, progesterone receptors and estrogen receptors, information on treatment (e.g., whether hormone therapy and/or chemotherapy was received), and information on a survival outcome (e.g., the time from primary surgery to death or censoring).

In some examples, common features between patients are similar when they have the same or similar values for each patient. For instance, in an example, two patients with the same age have similar age features. Further, in some examples, a first common feature value of a first patient differs from a second value of the same common feature of a second patient, but the first common feature value and second common feature value are within a defined range of each other such that they are considered to be “similar”. For instance, in an example, the first value and second value are considered to be similar when the first value is less than 10% different than the second value (e.g., if the second value is 100, the first value is similar if it is between 90-110). Alternatively, or additionally, the first value and second value are considered to be similar when the first value is within a defined quantity of units of the second value (e.g., if the second value is 100 and the defined quantity of units is 15, the first value is similar if it is between 85-115). Still further, in some examples, a defined quantity or percentage of patients are considered similar to a target patient based on the common feature values of the defined quantity of patients being the closest to the common feature value of the target patient (e.g., the 5% of possible patients with age values closest to the age value of the target patient are considered similar to the target patient). In other examples, other methods of determining that two patients are similar are used without departing from the description.

In some examples, the drug is administered to the patients in the target group of patients and those patients are compared with the synthetic patients representing, or corresponding to, patients in the control group of patients. Based on this comparison between patients in the target group and patients in the control group, efficacy of the drug is determined. The efficacy may be determined by determining a time to event outcome of the synthetic patient for each patient in the control group of patients. For example, the synthetic patient for each patient in the control group of patients is created on an outcome scale or on log scale. Penalizing the distance comprises using the variance penalty and a covariance penalty.

The target group of patients and the control group of patients are created from data associated with the plurality of patients by following a biased sampling scheme comprising: fitting a cox proportional hazards model using all covariates on the plurality of patients; predicting, using the cox proportional hazards model, an expected median survival time for each patient; and based on the expected median survival time, splitting the plurality of patients into the target group of patients and the control group of patients, wherein the target group of patients have the expected median survival time above a threshold (e.g., more than 2 years and the like).

In some examples, another synthetic patient may be created for each patient in the target group of patients. These other synthetic patients will have a common feature similar to a particular patient in the control group of patients. The drug may be administered to a subset of the target group of patients and the subset of the target group of patients who are administered the drug may be compared with the other synthetic patient for each patient in the subset of the target group of patients. Based on this comparison, the efficacy of the drug may be determined without actually needing placebo patients for the clinical trials.

FIG. 1 is a block diagram 100 illustrating an example synthetic control arm 108 as a solution for creating synthetic controls in survival analysis. Thus, there is no need to randomly select eligible patients (e.g., eligible patient 102) to receive the new treatment. Thus, all eligible patients receive the treatment as part of the single-armed trial 104. The real-world data 110 (e.g., from hospitals) is routinely collected and a machine learning (ML) model (such as a large learning model (LLM)) is applied by the synthetic control arms (SCA) generator 112 to create synthetic (e.g., virtual) patients in the control arm 108 that historically received standard of care (or a placebo). These synthetic patients are compared with the patients who will receive the new treatment to determine the effect 114 of the new treatment.

In some examples, the model of the SCA generator 112 is configured to obtain one or more features of an eligible patient 102 and, based on those one or more features, generate one or more synthetic patients associated with that eligible patient 102 using the real-world data 110. For instance, in an example, the SCA generator 112 identifies other patients in the real-world data 110 that are similar to the eligible patient 102 at least with respect to the one or more obtained features. The results over time associated with the identified other patients are weighed and averaged as described herein to generate a synthetic patient that is similar to the eligible patient 102. The averaged results over time of the synthetic patient are compared to the results over time for the eligible patient 102 to generate data that indicates a treatment effect 114. This process is described in greater detail herein.

In some examples, a user interface (UI) shows the efficacy or the determined effect of the new treatment. If the effect or efficacy of the new treatment is above a threshold, the efficacy may be notified in a first portion of the UI. However, if the effect or efficacy of the new treatment is below the threshold, the efficacy may be notified in a second portion of the UI. The notification may comprise text, icon, or any other form of notification. As the real-world data is routinely getting collected, the notification (e.g., an icon) of the effect or efficacy of the new treatment may dynamically shift, or otherwise be automatically moved, from the first portion of the UI to the second portion of the UI and vice versa.

Additionally, or alternatively, in some examples, the disclosed system is configured to automatically generate reports that describe the generated synthetic patients and/or describe the treatment effects 114 that are determined during operation of the system. In some such examples, the system automatically generates such a report and displays it on a UI such as the UI described above. Further, in some examples, the system dynamically updates the generated report during operation and, as a result of those dynamic updates, the display of the report on the UI is adjusted, changed, moved, or otherwise updated to reflect the dynamic updates.

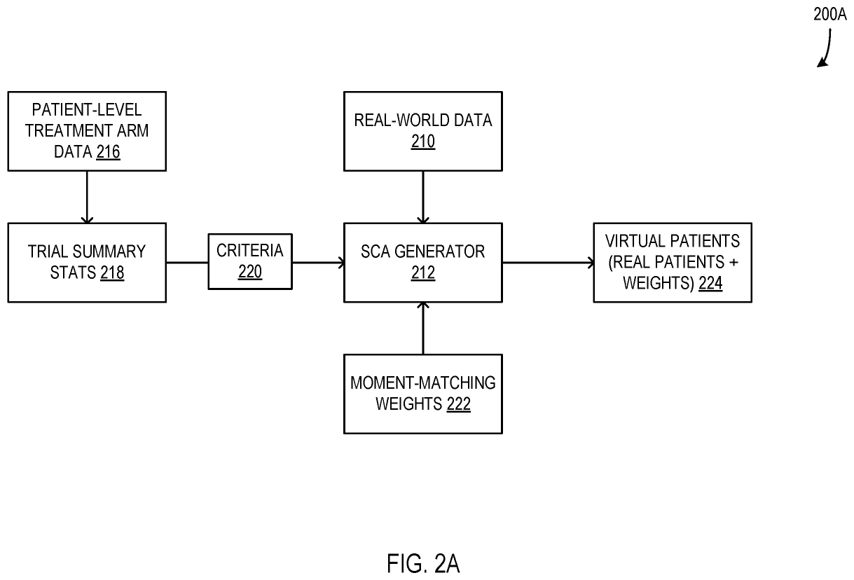

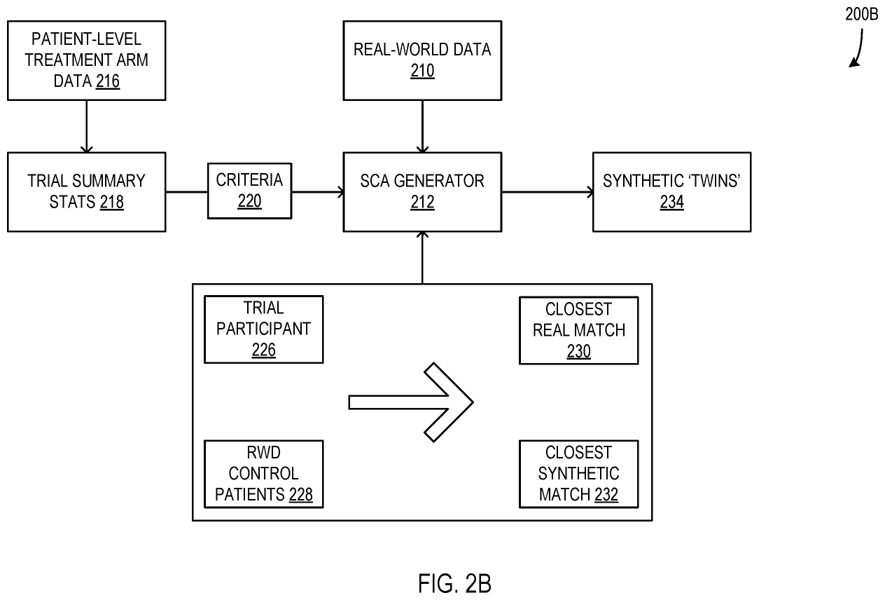

FIG. 2A is a block diagram 200A illustrating an example implementation generating synthetic control arms from aggregate inputs. Patient-level treatment arm data 216 is input and trial summary statistics 218 are extracted from this input data. Inclusion exclusion criteria 220 are applied to the trial summary statistics 218 after which the SCA generator 212 processes the real-world data 210 and the inclusion exclusion applied trial summary statistics 218 to determine moment matching weights 222. As a result, the weighted trial mean becomes similar to the real-world data mean. Based on this processing, the SCA generator 212 outputs synthetic patients 224 whose characteristics are similar to real patients and weights to be applied to them.

FIG. 2B is a block diagram 200B illustrating an example implementation generating synthetic control arms from patient-level inputs. Patient-level treatment arm data 216 are input and inclusion exclusion criteria 220 are applied to trial summary statistics 218 of the patient-level treatment arm data 216. After which the SCA generator 212 processes the real-world data 210 and the inclusion exclusion applied patient-level treatment arm data to determine synthetic twins 234 which are non-trivial with time-to-event outcomes. Based on this processing, the SCA generator outputs synthetic twins 234. In some examples, the SCA generator 212 analyzes data associated with a trial participant 226 and real-world data control patients 228 to identify a closest real match 230 between the trial participant 226 and the control patients 228 and to generate a closest synthetic match 232 to the trial participant 226 that still maintains the data patterns of the real-world data 210, as described herein.

In some examples, the generation of synthetic controls for survival analyses leads to bias because the distribution of the synthetic control patients and/or other units differs from the distribution of the real control patients and/or units (e.g., depending on the behavior of the weight vectors for each synthetic unit). Because of these biases, in some examples, the generation of the synthetic controls includes penalizing the weight vectors based on the deviation of the synthetic controls from the distribution of the associated real control units. For instance, in some examples, the weight vector generation process includes a tradeoff of the fit in the input space (e.g., extrapolation bias) with the bias incurred due to artificially low variance of synthetic control units. To achieve this tradeoff, in some examples, the variance of the synthetic control outcome is maximized as illustrated below in equation 1.

$${w}_{*} = arg {min}_{w : 0 \leq {w}_{i} \leq 1 , {\sum }_{i} {w}_{i} = 1} {‖ {X}_{*} - {\sum }_{j = 1}^{m} {w}_{j} {X}_{j} ‖}^{2} - {λ}_{var} {‖ w ‖}^{2} \qquad ( 1 )$$

In equation 1, the weight vectors we are generated by minimizing the difference between the covariates of a target treated unit X, and the covariates of real control units Σⱼ₌₁^{m}wⱼXⱼ, wherein that portion of the equation is penalized using −λᵥₐᵣ∥w|², which includes a variance penalty Avar multiplied by the matrix norm of the weight vectors squared ∥w|². The weight vectors squared ∥w|² is maximized when all weight is given to a single donor unit. Thus, the objective of equation 1 interpolates between a perfect synthetic control (when λᵥₐᵣ=0) and a “nearest neighbor” matching estimator (when λᵥₐᵣ=00) at its extremes. Because ∥w|² grows as weight is assigned less uniformly, this objective encourages more sparsity in the weights at intermediate values, which, as a side effect, also leads to less interpolation bias. Further, in some examples, if the goal is to correct for or otherwise minimize the covariance between units, this can be achieved by minimizing wₖ^{T}wₗ for l≠k, i.e. adding a term +λ_{cov}Σₖ₌₁ⁿΣ_{l>k}wₖ^{T}wₗ, which will reduce overlap between donor units.

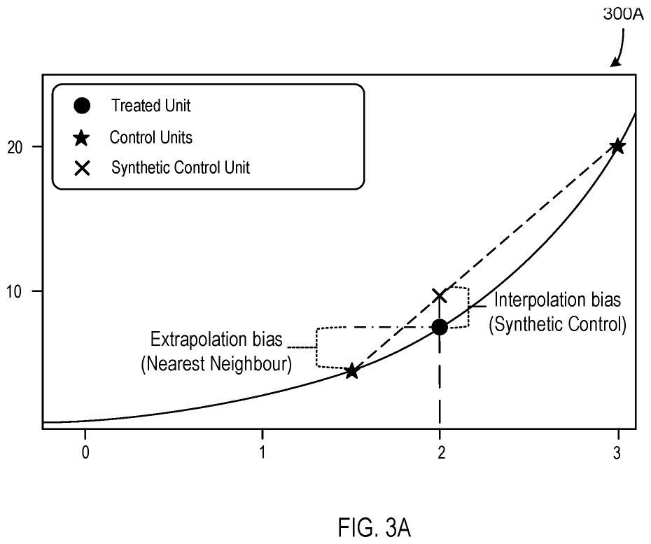

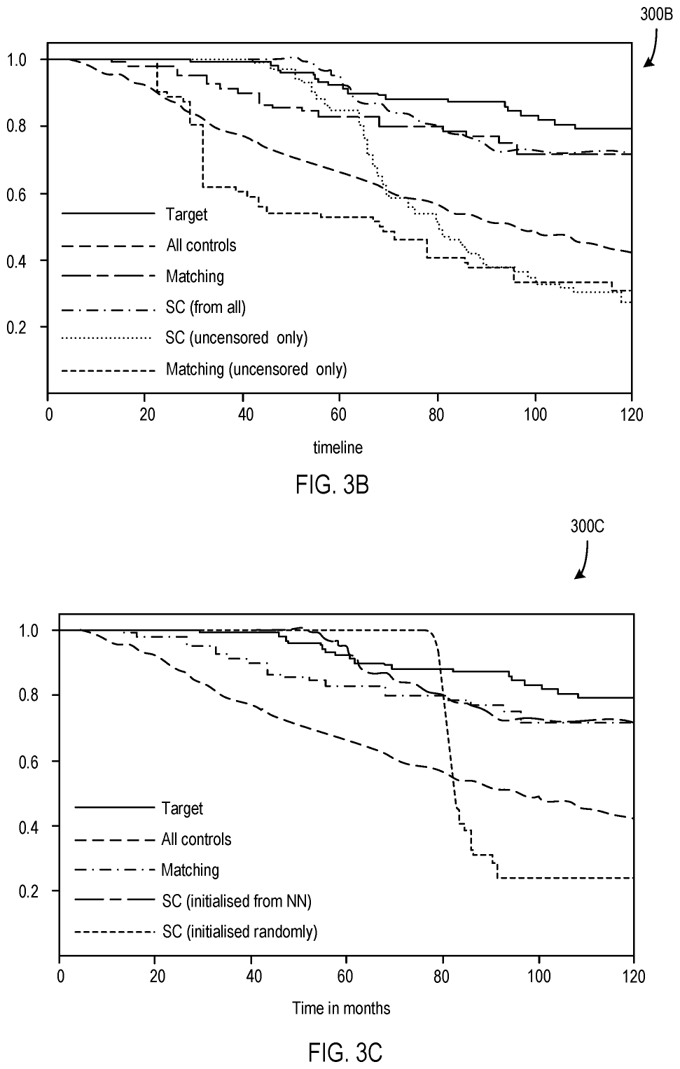

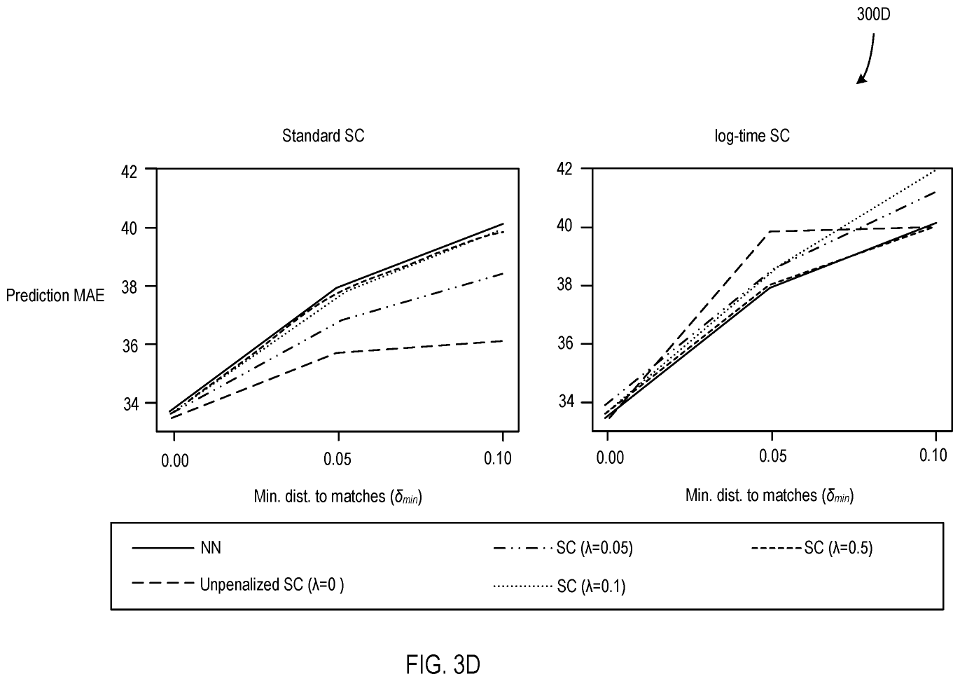

FIG. 3A illustrates an example graph 300A showing that interpolation bias is high on the non-linear part of the exponential function. The inclusion of the variance penalty portion of the above equation 1 adjusts the synthetic control unit to be closer to the treated unit, thereby reducing the interpolation bias. In some such cases, the use of the variance penalty may also increase the extrapolation bias in the same processes, but the reduced interpolation bias may still result in the resulting synthetic control being more accurate overall.

FIG. 3B illustrates an example graph 300B showing censoring bias. In some examples, the data used to generate the synthetic control units as described herein is censored. If the data is not censored at random (CAR), biases can be introduced that reduces the accuracy of the generated synthetic control units. For instance, in an example associated with the treatment of patients over prolonged periods of time, longer times are naturally more likely to be censored as follow-up is limited (e.g., only 50-70% of individuals experience an event by 120 months). Constructing matched survival curves from only uncensored individuals, be it through matching or through synthetic control, therefore leads to downward bias, as can be seen in FIG. 3B. In particular, the curve associated with synthetic control using all data is much closer to the target curve than the curve associated with synthetic control using only uncensored data. To avoid such biases, in some examples, the synthetic control weights are applied to both the censored times Tⱼ^{-=min(T,C)} and the event indicators Eⱼ=1{T<C} and then heuristically censor the synthetic control units going forward (e.g., with Σⱼ₌₁^{m}wⱼEⱼ<0.5).

FIG. 3C illustrates an example graph 300C showing initialization effects of bias. In some examples, the synthetic control generation process includes initializing the weights to match a “nearest neighbor” to the target treated unit (e.g., a real control unit that is the most similar to the target treated unit). The initialization of the weights to the “nearest neighbor” is demonstrated as being more accurate to the target curve than when the weights are initialized to random values. In some such examples, solutions are not unique and the random value initialization results in a solution with many more contributors such that the survival times are too concentrated around the mean.

FIG. 3D includes example graphs 300D illustrating performance of mean absolute error (MAE) of predicting the Restricted Mean Survival Time (RMST). The FIG. 3D provides comparisons between different variance penalties and between using standard time and log-time during the generation of the synthetic controls. Further, each SC curve is compared to “nearest neighbor” matching curve. As shown, in some examples, the generation of synthetic controls can be done using standard time or log-time and that choice may be based on specific features of the dataset that is being analyzed.

Additionally, FIG. 3D demonstrates that using small variance penalties leads to synthetic control cohorts with curves between the extremes of matching techniques and unpenalized synthetic control generation. Thus, they perform better in terms of distribution metrics than unpenalized synthetic control generation and better in terms of prediction metrics than matching techniques.

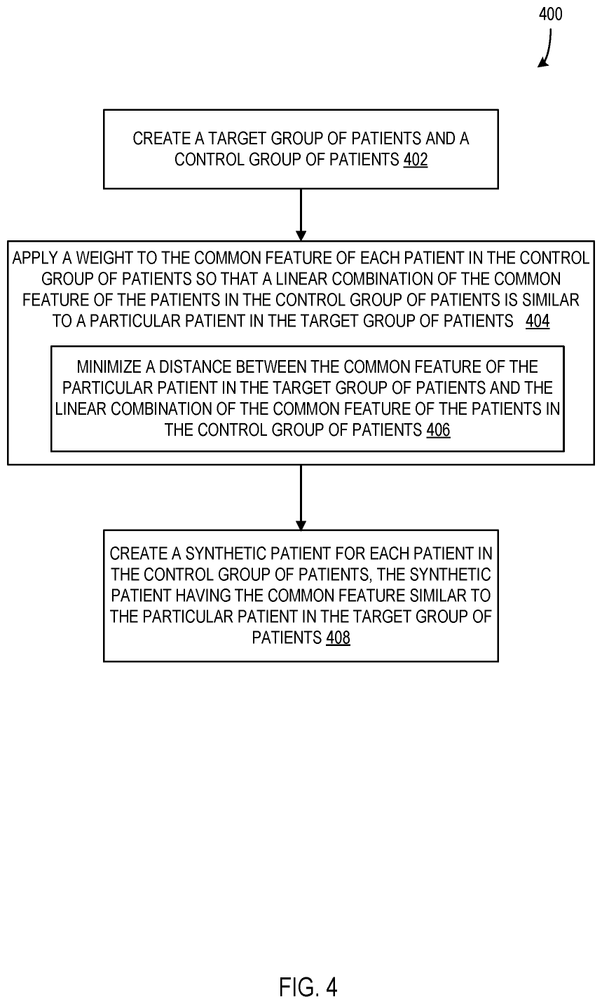

FIG. 4 illustrates an example flow chart implementing aspects of the disclosure for creating synthetic controls in survival analysis. At 402, a target group of patients and a control group of patients are created from data associated with a plurality of patients. The target group of patients includes patients to whom a drug is to be administered, and the control group of patients includes patients to whom the drug is not administered. Each patient in the target group of patients and the control group of patients have a common feature. At 404, a weight is applied to the common feature of each patient in the control group of patients, so that a linear combination of the common feature of the patients in the control group of patients becomes similar to a particular patient in the target group of patients. At 406, the weight that is applied to the common feature of each patient in the control group of patients is selected, determined, generated, or the like, so as to minimize a distance between the common feature of the particular patient in the target group of patients and the linear combination of the common feature of the patients in the control group of patients. The distance is minimized by penalizing the distance using a variance penalty and/or a covariance penalty.

At 408, a synthetic patient is created for each patient in the control group of patients such that the synthetic patient has the common feature similar to the particular patient in the target group of patients. Because the synthetic patient has the common feature similar to the particular patient in the target group of patients, the characteristics of the synthetic patient are similar to the characteristics of the particular patient in the target group of patients. As such, without needing a placebo patient, the synthetic patients function as the placebo patients for the purposes of clinical trials.

In some examples, minimizing the distance between the common feature of the particular patient in the target group of patients and the linear combination of the common feature of the patients in the control group of patients includes initializing the applied weight or weights to correspond to the common feature(s) of a nearest neighbor patient in the control group of patients to the particular patient (e.g., the patient in the control group of patients that is most similar to the particular patient at least with respect to the common feature(s) being analyzed).

Further, in some examples, the data associated with the plurality of patients includes censored data. In order to manage the censored data, the weight is applied to censored time data and censored event indicators of the censored data and then the created synthetic patients are heuristically censored based on the censored data.

Additionally, or alternatively, in some examples, the creation of the synthetic patients is performed using standard time techniques and/or log-time techniques as described herein.

While the examples of the disclosure have been described in healthcare context (e.g., for clinical trials of new drugs or trials for medical procedures), examples of the disclosure may be implemented to compare an intervention in supply chain management, and to build a synthetic control arm for the data obtained from the supply chain. For example, if a user wants to perform A/B testing for addressing and mitigating disruptions to a supply chain, there are two groups to be compared and an experiment to be run with the two groups. Data for one of the groups is not available, a control group may be synthetically built using examples of the disclosure. Thus, different response strategies may be tested, different inventory level scenarios may be tested to see how much merchandise to keep on hand, and/or experiments with alternative transportation and logistics may be performed.

In some examples of variance-penalized synthetic control, the inputs to the system include covariates of the target treated unit, covariates of the uncensored control units, time-to-event outcomes of the uncensored control units, and a variance penalty. The output of the system is a synthetic control unit, constructed on an outcome scale or on a log-scale.

In some examples of variance-covariance penalized synthetic controls, the inputs to the system include covariates of the target treated unit, covariates of the uncensored control units, time-to-event outcomes of the uncensored control units, a variance penalty, and a covariance penalty. The outputs of the system are synthetic control units, constructed on an outcome scale or on a log-scale.

### Exemplary Operating Environment

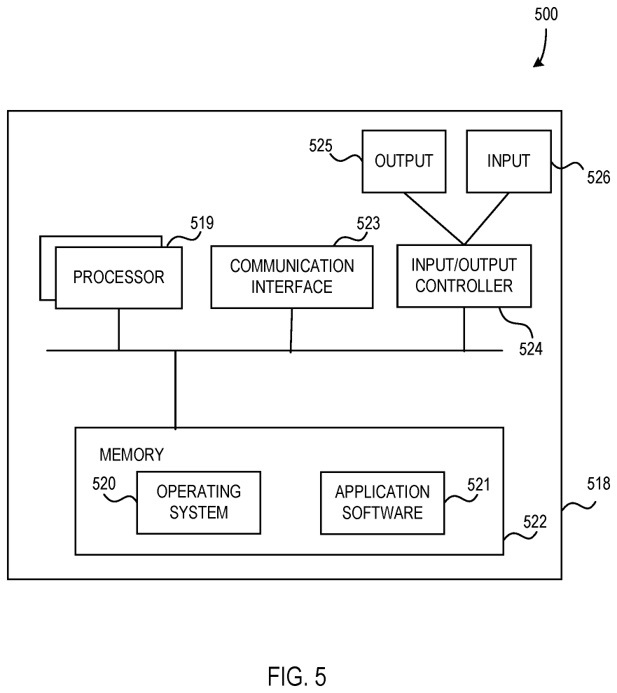

The present disclosure is operable with a computing apparatus according to an embodiment as a functional block diagram 500 in FIG. 5. In an example, components of a computing apparatus 518 are implemented as a part of an electronic device according to one or more embodiments described in this specification. The computing apparatus 518 comprises one or more processors 519 which may be microprocessors, controllers, or any other suitable type of processors for processing computer executable instructions to control the operation of the electronic device. Alternatively, or in addition, the processor 519 is any technology capable of executing logic or instructions, such as a hard-coded machine. In some examples, platform software comprising an operating system 520 or any other suitable platform software is provided on the apparatus 518 to enable application software 521 to be executed on the device. In some examples, the functionality of creating synthetic controls in survival analysis is accomplished by software, hardware, and/or firmware.

In some examples, computer executable instructions are provided using any computer-readable media that is accessible by the computing apparatus 518. Computer-readable media include, for example, computer storage media such as a memory 522 and communications media. Computer storage media, such as a memory 522, include volatile and non-volatile, removable, and non-removable media implemented in any method or technology for storage of information such as computer readable instructions, data structures, program modules or the like. Computer storage media include, but are not limited to, Random Access Memory (RAM), Read-Only Memory (ROM), Erasable Programmable Read-Only Memory (EPROM), Electrically Erasable Programmable Read-Only Memory (EEPROM), persistent memory, phase change memory, flash memory or other memory technology, Compact Disk Read-Only Memory (CD-ROM), digital versatile disks (DVD) or other optical storage, magnetic cassettes, magnetic tape, magnetic disk storage, shingled disk storage or other magnetic storage devices, or any other non-transmission medium that can be used to store information for access by a computing apparatus. In contrast, communication media may embody computer readable instructions, data structures, program modules, or the like in a modulated data signal, such as a carrier wave, or other transport mechanism. As defined herein, computer storage media does not include communication media. Therefore, a computer storage medium does not include a propagating signal. Propagated signals are not examples of computer storage media. Although the computer storage medium (the memory 522) is shown within the computing apparatus 518, it will be appreciated by a person skilled in the art, that, in some examples, the storage is distributed or located remotely and accessed via a network or other communication link (e.g., using a communication interface 523).

Further, in some examples, the computing apparatus 518 comprises an input/output controller 524 configured to output information to one or more output devices 525, for example a display or a speaker, which are separate from or integral to the electronic device. Additionally, or alternatively, the input/output controller 524 is configured to receive and process an input from one or more input devices 526, for example, a keyboard, a microphone, or a touchpad. In one example, the output device 525 also acts as the input device. An example of such a device is a touch sensitive display. The input/output controller 524 may also output data to devices other than the output device, e.g., a locally connected printing device. In some examples, a user provides input to the input device(s) 526 and/or receives output from the output device(s) 525.

The functionality described herein can be performed, at least in part, by one or more hardware logic components. According to an embodiment, the computing apparatus 518 is configured by the program code when executed by the processor 519 to execute the embodiments of the operations and functionality described. Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Program-specific Standard Products (ASSPs), System-on-a-chip systems (SOCs), Complex Programmable Logic Devices (CPLDs), Graphics Processing Units (GPUs).

At least a portion of the functionality of the various elements in the figures may be performed by other elements in the figures, or an entity (e.g., processor, web service, server, application program, computing device, or the like) not shown in the figures.

Although described in connection with an exemplary computing system environment, examples of the disclosure are capable of implementation with numerous other general purpose or special purpose computing system environments, configurations, or devices.

Examples of well-known computing systems, environments, and/or configurations that are suitable for use with aspects of the disclosure include, but are not limited to, mobile or portable computing devices (e.g., smartphones), personal computers, server computers, hand-held (e.g., tablet) or laptop devices, multiprocessor systems, gaming consoles or controllers, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, and the like. In general, the disclosure is operable with any device with processing capability such that it can execute instructions such as those described herein. Such systems or devices accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions, or the specific components or modules illustrated in the figures and described herein. Other examples of the disclosure include different computer-executable instructions or components having more or less functionality than illustrated and described herein.

In examples involving a general-purpose computer, aspects of the disclosure transform the general-purpose computer into a special-purpose computing device when configured to execute the instructions described herein.

An example system comprises a processor; and a memory comprising computer program code, the memory and the computer program code configured to cause the processor to: create a target group of patients and a control group of patients from data associated with a plurality of patients, the target group of patients comprising patients to whom a drug is to be administered, the control group of patients comprising patients to whom the drug is not administered, each patient in the target group of patients and the control group of patients having a common feature; apply a weight to the common feature of each patient in the control group of patients so that a linear combination of the common feature of the patients in the control group of patients becomes similar to a particular patient in the target group of patients, applying the weight comprising: minimizing a distance between the common feature of the particular patient in the target group of patients and the linear combination of the common feature of the patients in the control group of patients, the distance being minimized by penalizing the distance using a variance penalty; and create a synthetic patient for each patient in the control group of patients, the synthetic patient having the common feature similar to the particular patient in the target group of patients.

An example computerized method comprises creating a target group of patients and a control group of patients from data associated with a plurality of patients, the target group of patients comprising patients to whom a drug is to be administered, the control group of patients comprising patients to whom the drug is not administered, each patient in the target group of patients and the control group of patients having a common feature; applying a weight to the common feature of each patient in the control group of patients so that a linear combination of the common feature of the patients in the control group of patients becomes similar to a particular patient in the target group of patients, applying the weight comprising: minimizing a distance between the common feature of the particular patient in the target group of patients and the linear combination of the common feature of the patients in the control group of patients, the distance being minimized by penalizing the distance using a variance penalty; and creating a synthetic patient for each patient in the control group of patients, the synthetic patient having the common feature similar to the particular patient in the target group of patients.

One or more computer storage media having computer-executable instructions that, upon execution by a processor, case the processor to at least: create a target group of patients and a control group of patients from data associated with a plurality of patients, the target group of patients comprising patients on whom a medical procedure is to be performed, the control group of patients comprising patients on whom the medical procedure is not performed, each patient in the target group of patients and the control group of patients having a common feature; apply a weight to the common feature of each patient in the control group of patients so that a linear combination of the common feature of the patients in the control group of patients becomes similar to a particular patient in the target group of patients, applying the weight comprising: minimizing a distance between the common feature of the particular patient in the target group of patients and the linear combination of the common feature of the patients in the control group of patients, the distance being minimized by penalizing the distance using a variance penalty; and create a synthetic patient for each patient in the control group of patients, the synthetic patient having the common feature similar to the particular patient in the target group of patients.

Alternatively, or in addition to the other examples described herein, examples include any combination of the following:
further comprising: causing the drug to be administered to the target group of patients; comparing the target group of patients who are administered the drug with the synthetic patient for each patient in the control group of patients; and based on the comparison, determining an efficacy of the drug.
further comprising creating another synthetic patient for each patient in the target group of patients, the other synthetic patient having the common feature similar to a particular patient in the control group of patients.
further comprising: administering the drug to a subset of the target group of patients; comparing the subset of the target group of patients who are administered the drug with the other synthetic patient for each patient in the subset of the target group of patients; and based on the comparison, determining an efficacy of the drug.
further comprising determining a time to event outcome of the synthetic patient for each patient in the control group of patients.
wherein the synthetic patient for each patient in the control group of patients is created on an outcome scale or on log scale.
wherein the penalizing the distance comprises using the variance penalty and a covariance penalty.
wherein the target group of patients and the control group of patients are created from data associated with the plurality of patients by following a biased sampling scheme, the biased sampling scheme comprising: fitting a cox proportional hazards model using all covariates on the plurality of patients; predicting, using the cox proportional hazards model, an expected median survival time for each patient; and based on the expected median survival time, splitting the plurality of patients into the target group of patients and the control group of patients, wherein the target group of patients have the expected median survival time above a threshold.
wherein minimizing the distance between the common feature of the particular patient in the target group of patients and the linear combination of the common feature of the patients in the control group of patients further includes initializing the applied weight to correspond with the common feature of a nearest neighbor patient in the control group of patients to the particular patient.
wherein the data associated with the plurality of patients includes censored data; wherein applying the weight to the common feature of each patient in the control group of patients so that a linear combination of the common feature of the patients in the control group of patients becomes similar to a particular patient in the target group of patients further includes applying the weight to censored time data and censored event indicators of the censored data; and wherein the created synthetic patients are heuristically censored based on the censored data.
wherein creating the synthetic patient for each patient in the control group of patients includes at least one of the following: creating the synthetic patient for each patient in the control group of patients in standard time; and creating a synthetic patient for each patient in the control group of patients in log-time.

Any range or device value given herein may be extended or altered without losing the effect sought, as will be apparent to the skilled person.

Examples have been described with reference to data monitored and/or collected from the users (e.g., user identity data with respect to profiles). In some examples, notice is provided to the users of the collection of the data (e.g., via a dialog box or preference setting) and users are given the opportunity to give or deny consent for the monitoring and/or collection. The consent takes the form of opt-in consent or opt-out consent.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items.

The embodiments illustrated and described herein as well as embodiments not specifically described herein but within the scope of aspects of the claims constitute an exemplary means for creating a target group of patients and a control group of patients from data associated with a plurality of patients, the target group of patients comprising patients to whom a drug is to be administered, the control group of patients comprising patients to whom the drug is not administered, each patient in the target group of patients and the control group of patients having a common feature; exemplary means for applying a weight to the common feature of each patient in the control group of patients so that a linear combination of the common feature of the patients in the control group of patients becomes similar to a particular patient in the target group of patients, applying the weight comprising: exemplary means for minimizing a distance between the common feature of the particular patient in the target group of patients and the linear combination of the common feature of the patients in the control group of patients, the distance being minimized by penalizing the distance using a variance penalty; and exemplary means for creating a synthetic patient for each patient in the control group of patients, the synthetic patient having the common feature similar to the particular patient in the target group of patients.

The term “comprising” is used in this specification to mean including the feature(s) or act(s) followed thereafter, without excluding the presence of one or more additional features or acts.

In some examples, the operations illustrated in the figures are implemented as software instructions encoded on a computer readable medium, in hardware programmed or designed to perform the operations, or both. For example, aspects of the disclosure are implemented as a system on a chip or other circuitry including a plurality of interconnected, electrically conductive elements.

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing a particular operation before, contemporaneously with, or after another operation is within the scope of aspects of the disclosure.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A system comprising:
a processor; and
a memory comprising computer program code, the memory and the computer program code configured to cause the processor to:
create a target group of patients and a control group of patients from data associated with a plurality of patients, the target group of patients comprising patients to whom a drug is to be administered, the control group of patients comprising patients to whom the drug is not administered, each patient in the target group of patients and the control group of patients having a common feature;
apply a weight to the common feature of each patient in the control group of patients so that a linear combination of the common feature of the patients in the control group of patients becomes similar to a particular patient in the target group of patients, wherein applying the weight comprises:
minimizing a distance between the common feature of the particular patient in the target group of patients and the linear combination of the common feature of the patients in the control group of patients, the distance being minimized by penalizing the distance using a variance penalty; and
create a synthetic patient for each patient in the control group of patients, the synthetic patient having the common feature similar to the particular patient in the target group of patients.

2. The system of claim 1, wherein the memory and the computer program code are configured to further cause the processor to:
cause the drug to be administered to the target group of patients;
compare the target group of patients who are administered the drug with the synthetic patient for each patient in the control group of patients; and
based on the comparison, determine an efficacy of the drug.

3. The system of claim 1, wherein minimizing the distance between the common feature of the particular patient in the target group of patients and the linear combination of the common feature of the patients in the control group of patients further includes initializing the applied weight to correspond with the common feature of a nearest neighbor patient in the control group of patients to the particular patient.

4. The system of claim 1, wherein the data associated with the plurality of patients includes censored data;
wherein applying the weight to the common feature of each patient in the control group of patients so that a linear combination of the common feature of the patients in the control group of patients becomes similar to a particular patient in the target group of patients further includes applying the weight to censored time data and censored event indicators of the censored data; and
wherein the created synthetic patients are heuristically censored based on the censored data.

5. A computerized method for creating synthetic controls in survival analysis, the computerized method comprising:
creating a target group of patients and a control group of patients from data associated with a plurality of patients, the target group of patients comprising patients to whom a drug is to be administered, the control group of patients comprising patients to whom the drug is not administered, each patient in the target group of patients and the control group of patients having a common feature;
applying a weight to the common feature of each patient in the control group of patients so that a linear combination of the common feature of the patients in the control group of patients becomes similar to a particular patient in the target group of patients, wherein applying the weight comprises:
minimizing a distance between the common feature of the particular patient in the target group of patients and the linear combination of the common feature of the patients in the control group of patients, the distance being minimized by penalizing the distance using a variance penalty; and
determining the weight to be applied based on the minimizing; and
creating a synthetic patient for each patient in the control group of patients, the synthetic patient having the common feature similar to the particular patient in the target group of patients.

6. The computerized method of claim 5, further comprising:
causing the drug to be administered to the target group of patients;
comparing the target group of patients who are administered the drug with the synthetic patient for each patient in the control group of patients; and
based on the comparison, determining an efficacy of the drug.

7. The computerized method of claim 5, further comprising creating another synthetic patient for each patient in the target group of patients, the other synthetic patient having the common feature similar to a particular patient in the control group of patients.

8. The computerized method of claim 7, further comprising:
administering the drug to a subset of the target group of patients;
comparing the subset of the target group of patients who are administered the drug with the other synthetic patient for each patient in the subset of the target group of patients; and
based on the comparison, determining an efficacy of the drug.

9. The computerized method of claim 5, further comprising determining a time to event outcome of the synthetic patient for each patient in the control group of patients.

10. The computerized method of claim 5, wherein the synthetic patient for each patient in the control group of patients is created on an outcome scale or on log scale.

11. The computerized method of claim 5, wherein the penalizing the distance comprises using the variance penalty and a covariance penalty.

12. The computerized method of claim 5, wherein the target group of patients and the control group of patients are created from data associated with the plurality of patients by following a biased sampling scheme, the biased sampling scheme comprising:
fitting a cox proportional hazards model using all covariates on the plurality of patients;
predicting, using the cox proportional hazards model, an expected median survival time for each patient; and
based on the expected median survival time, splitting the plurality of patients into the target group of patients and the control group of patients, wherein the target group of patients have the expected median survival time above a threshold.

13. The computerized method of claim 5, wherein minimizing the distance between the common feature of the particular patient in the target group of patients and the linear combination of the common feature of the patients in the control group of patients further includes initializing the applied weight to correspond with the common feature of a nearest neighbor patient in the control group of patients to the particular patient.

14. The computerized method of claim 5, wherein the data associated with the plurality of patients includes censored data;
wherein applying the weight to the common feature of each patient in the control group of patients so that a linear combination of the common feature of the patients in the control group of patients becomes similar to a particular patient in the target group of patients further includes applying the weight to censored time data and censored event indicators of the censored data; and
wherein the created synthetic patients are heuristically censored based on the censored data.

15. The computerized method of claim 5, wherein creating the synthetic patient for each patient in the control group of patients includes at least one of the following:
creating the synthetic patient for each patient in the control group of patients in standard time; and
creating a synthetic patient for each patient in the control group of patients in log-time.

16. A computer storage medium has computer-executable instructions that, upon execution by a processor, cause the processor to at least:
create a target group of patients and a control group of patients from data associated with a plurality of patients, the target group of patients comprising patients on whom a medical procedure is to be performed, the control group of patients comprising patients on whom the medical procedure is not performed, each patient in the target group of patients and the control group of patients having a common feature;
apply a weight to the common feature of each patient in the control group of patients so that a linear combination of the common feature of the patients in the control group of patients becomes similar to a particular patient in the target group of patients, wherein applying the weight comprising:
minimizing a distance between the common feature of the particular patient in the target group of patients and the linear combination of the common feature of the patients in the control group of patients, the distance being minimized by penalizing the distance using a variance penalty; and
create a synthetic patient for each patient in the control group of patients, the synthetic patient having the common feature similar to the particular patient in the target group of patients.

17. The computer storage medium of claim 16, wherein the computer-executable instructions, upon execution by the processor, further cause the processor to at least create another synthetic patient for each patient in the target group of patients, the other synthetic patient having the common feature similar to a particular patient in the control group of patients.

18. The computer storage medium of claim 17, wherein the computer-executable instructions, upon execution by the processor, further cause the processor to:
cause the medical procedure to be performed on a subset of the target group of patients;
compare the subset of the target group of patients on whom the medical procedure is performed with the other synthetic patient for each patient in the subset of the target group of patients; and
based on the comparison, determine an efficacy of the medical procedure.

19. The computer storage medium of claim 18, wherein the computer-executable instructions, upon execution by the processor, further cause the processor to:
generate a report associated with the created synthetic patients and the determined efficacy of the medical procedure;
display the generated report on a user interface (UI), including displaying information associated with the determined efficacy in a first location of the UI based on the determined efficacy exceeding a threshold;
update the generated report dynamically based on determining additional efficacy information, wherein a value of the determined efficacy is changed based on the update; and
move the information associated with the determined efficacy to a second location of the UI based on the changed value of the determined efficacy being less than the threshold.

20. The computer storage medium of claim 16, wherein minimizing the distance between the common feature of the particular patient in the target group of patients and the linear combination of the common feature of the patients in the control group of patients further includes initializing the applied weight to correspond with the common feature of a nearest neighbor patient in the control group of patients to the particular patient.
